(19) United States
(12) Patent Application Publication
(10) Pub. No.: US 2020/0299139 A1
(43) Pub. Date: Sep. 24, 2020

(54) COMPOSITIONS AND METHODS FOR USING ACTIVATED CARBON PARTICLES FOR PURIFICATION OF NUCLEIC ACIDS

(71) Applicant: Virongy L.L.C., Manassas, VA (US)

(72) Inventor: Yuntao WU, Manassas, VA (US)

(73) Assignee: Virongy L.L.C.

(21) Appl. No.: 16/823,704

(22) Filed: Mar. 19, 2020

Related U.S. Application Data
(60) Provisional application No. 62/820,311, filed on Mar. 19, 2019.

(51) Int. Cl. C01B 32/312; C12N 15/10

(52) U.S. Cl. CPC C01B 32/312; C12N 2500/05; C12N 15/1003

(57) ABSTRACT

Compositions and methods to improve the process of nucleic aids purification from bacterial, prokaryotic and eukaryotic cells through the use of activated carbon particles.

### CROSS-REFERENCE TO RELATED APPLICATIONS

This application claims priority from U.S. Provisional Application No. 62/820,311, filed Mar. 19, 2019, the entire disclosure of which is hereby incorporated by reference.

### FIELD

The present disclosure relates to compositions and methods to improve the process of nucleic aids purification from bacterial, prokaryotic and eukaryotic cells through the use of activated carbon particles. Provided herein are methodology, compositions, and the like for using activated carbon particles to remove impurities in the purification of nucleic acids.

### INTRODUCTION

Nucleic Acids Purification Methods: Nucleic Acids such as plasmid DNA, genomic DNA, and RNA are frequently purified from bacterial and eukaryotic cells through the use of various methods such as alkaline-SDS lysis method, mini-column purification method, phenol-chloroform extraction method, and Boom method et al. The Alkaline lysis method is frequently used to isolate plasmid DNA from bacteria [#3656]. The procedure uses alkaline solution, consisting of the detergent sodium dodecyl sulfate (SDS) and sodium hydroxide (NaOH), to disrupt cell membranes and denature both chromosomal and plasmid DNA. Following cell lysis, potassium acetate is added to renature plasmid DNA, but not chromosomal DNA which is removed by centrifugation. The plasmid-containing supernatant is further purified by ethanol precipitation or DNA binding mini-columns.

Most DNA binding mini-column uses a solid phase of silica to bind to DNA under certain conditions such as the presence of chaotropic agents (e.g. guanidinium thiocyanate or guanidinium hydrochloride). Under these conditions, nucleic acids bind to the silica gel membrane inside the spin column. Cell debris, contaminating proteins, or other impurities are washed away by adding a wash buffer to the column. DNA is finally eluted from the column by adding an elution buffer or simply water.

In comparison with the mini-column method, a traditional way to purify nucleic acids is to use phenol-chloroform extraction. In this method, DNA solution is mixed with an equal volume of a phenol: chloroform solution. The mixture is then centrifuged and two distinct phases are formed. The nucleic acids-containing aqueous phase is on the top and the proteins and hydrophobic lipids-containing organic phase is at the bottom. The nucleic acids-containing aqueous phase is collected and further purified by ethanol precipitation. Recent years, the phenol-chloroform method is largely replaced by spin-column-based method for simple and quick nucleic acids purification, largely because of the development of the Boom Method for nucleic acids purification.

The Boom method (Boom nucleic acid extraction method) [#3655] is a solid phase extraction method for isolating nucleic acid through the use of silica beads or silica membranes which are capable of binding the nucleic acids in the presence of chaotropic substances. The method is developed into mini-spin columns for simple and quick nucleic acids purification, and became widely used.

Activated Carbon and its Application:

Activated carbon or activated charcoal is a form of carbon that is processed to have small, low-volume pores with high surface area for adsorption of chemical substances. It is produced from carbonaceous source materials such as nutshells, peat, wood, coal, or petroleum pitch. It is often produced by either physical or chemical activation. In physical activation, the carbonaceous material is pyrolyzed using hot gases (600-990° C. argon or nitrogen gas), followed by oxidation by exposure to oxidizing atmospheres (oxygen or air steam) at temperatures around 600-1200° C. In chemical activation, the raw carbonaceous material is first impregnated with chemicals such as acid, strong base, or salt (e.g. phosphoric acid, potassium hydroxide, sodium hydroxide, calcium chloride, and zinc chloride). Then, the raw material is carbonized at lower temperatures (450-900° C.).

Activated carbon has been extensively used in pharmacy, industry, and microbiology to remove various contaminants from products, water, soil, and air with its adsorption capacity. For example, in metal finishing industry, activated carbon is widely used for purification of electroplating solutions, removing unwanted chemical breakdown product. Activated carbon is also used for treating food poisoning, drug overdoses, and diseases such as diarrhea, indigestion, and flatulence. In agriculture, activated carbon is used as a pesticide, animal feed additive, processing aid, and disinfectant. In organic winemaking, activated carbon is used as a processing agent to absorb brown color pigments from white grape concentrates. It is also used to filter impurities from liquors such as vodka and whiskey, significantly increasing purity as judged by order and taste. Activated carbon or nanoporous carbon materials are also used extensively in energy industry. The porous material can act like a sponge to adsorb and store natural gas and hydrogen gas. For gas purification, activated carbon is used to remove oil vapors, order, and other hydrocarbons. For nuclear boiling water reactor, activated carbon filers are used to retain radioactive gases within air vacuumed from the turbine condenser. For coal-fired power stations, activated carbon is infused with sulfur or iodine to trap mercury emissions.

Although activated carbon adsorbs many chemical substances, it does not bind well to certain chemicals, including alcohols, strong acids and bases, metals and most inorganics, such as lithium, sodium, iron, lead. The specific adsorption characteristics are strongly dependent on the composition of the surface functional groups. The surface of activated carbon is also capable of oxidation by atmospheric oxygen and oxygen plasma, steam, and also carbon dioxide, and ozone. Oxidation in the liquid is caused by a wide range of reagents such as HNO₃, H₂O₂, KMnO₄. Some of the chemical properties of activated carbon have been attributed to presence of the surface active carbon double bond.

### SUMMARY

In one aspect, provided is a method for using activated carbon to remove impurities present in bacterial cells, bacterial cell culture, or biological samples during the purification of bacterial genomic DNA or bacterial plasmid DNA.

In another aspect, provided is a method for using activated carbon particles to remove impurities present in animal cell culture, animal tissues samples, or animal fluid samples (e.g. blood, sperm samples) during the purification of genomic DNA, cytoplasmic DNA (e.g. mitochondrial DNA) or RNA.

In another aspect, provided is a method of formulating activated carbon for use in nucleic acid extraction, comprising (a) harvesting cells through centrifugation; (b) resuspending cells in lysis buffer; (c) adding activated carbon particles either before or after cell lysis; (d) removing cell debris and activated carbon particles by centrifugation; (e) applying DNA/RNA-containing supernatant to a DNA/RNA-binding column; (f) washing the column with washing buffer to remove cellular contaminants; and (g) eluting DNA with TE buffer or with H₂0.

In another aspect, provided is a kit comprising activated carbon, and one or more of DNA/RNA extracting buffers, DNA/RNA binding buffer, DNA/RNA-binding columns, column washing buffers, and DNA elution buffers.

Objects of Activated Carbon Particles

1) To remove impurities present in bacterial cell and cell lysate during the purification of bacterial genomic DNA or bacterial plasmid DNA.

2) To remove impurities present in animal cell and cell lysate during the purification of genomic DNA, cytoplasmic DNA (e.g. mitochondrial DNA) or RNA.

3) To remove impurities present in plant cell and cell lysate during the purification of genomic DNA or cytoplasmic DNA (e.g. chloroplast DNA)

### BRIEF DESCRIPTION OF THE DRAWINGS

FIG. 1: Activated carbon particles can remove impurities during the purification of plasmid DNA from E. coli. An equal amount of overnight bacterial cell culture (1.5 ml) was purified using a commercial Q miniSpin plasmid purification kit, or using a similar kit from Virong LLC (Virongy high purity miniSpin plasmid purification kit) in which activated charcoal particles were added to the bacterial cell lysates during purification. Following plasmid DNA purification, an equal amount of plasmid DNA, purified with the commercial Q miniSpin kit or with the Virongy miniSpin kit was digested with different amounts of E.coRI that was serially diluted (1:2 dilution). Following digestion, DNA was resolved by agarose gel electrophoresis. As shown, activated charcoal treatment leads to greater plasmid DNA purity that can be readily digested by E.coRI. At low E.coRI concentrations, the presence of impurities in the DNA purified from the Q miniSpin kit inhibits E.coRI activity, resulting in partial digestions.

FIG. 2: Activated carbon particles can remove impurities during plasmid DNA purification, leading to high purity DNA that can generate longer nucleotide read from DNA sequencing. An equal amount of overnight bacterial cell culture (1.5 ml) was purified using a commercial Q miniSpin plasmid purification kit, or using a similar kit from Virong LLC (Virongy high purity miniSpin plasmid purification kit) in which activated charcoal particles were added to bacterial cell lysates during plasmid purification. Following plasmid DNA purification, an equal amount of plasmid DNA, purified with the commercial Q miniSpin kit or with the Virongy miniSpin kit, was used as the template for DNA sequencing analysis. DNA purified with activated charcoal particles gave a sequence read 20 to 40 nucleotide longer than DNA purified with the commercial Q miniSpin kit (Average sequenced DNA length from activated charcoal/Virongy MiniSpin kit: 919±24.77; average sequenced DNA length from Q MiniSpin kit: 898±19.45; p=0.00196)

### DETAILED DESCRIPTION

The present disclosure relates to composition and methodology for use activated carbon or charcoal to improve the process of nucleic aids purification from bacterial, prokaryotic and eukaryotic cells. Provided herein are methodology, compositions, and the like for using activated carbon particles to remove impurities in the purification of nucleic acids.

In one embodiment, activated carbon particles are used to remove impurities present in bacterial cell lysate during the purification of bacterial genomic DNA or bacterial plasmid DNA.

In another embodiment, activated carbon particles are used to remove impurities present in animal cell lysate during the purification of genomic DNA, cytoplasmic DNA (e.g. mitochondrial DNA) or RNA.

In another embodiment, activated carbon particles are used to remove impurities present in plant cell lysate during the purification of genomic DNA or cytoplasmic DNA (e.g. chloroplast DNA)

Activated carbon refers to activated charcoal or any porous and nanoporous carbon materials that can be used to absorb chemicals and biochemical substances.

In other embodiments, provided is a kit comprising activated charcoal and one or more of DNA/RNA extracting buffers, DNA/RNA binding buffer, DNA/RNA-binding columns, column washing buffers, and DNA elution buffers.

All technical terms in this description are commonly used in biochemistry, molecular biology and immunology, respectively, and can be understood by those skilled in the field of this invention. Those technical terms can be found in: MOLECULAR CLONING: A LABORATORY MANUAL, 3rd ed., vol. 1-3, ed. Sambrook and Russel, Cold Spring Harbor Laboratory Press, Cold Spring Harbor, N.Y., 2001; CURRENT PROTOCOLS IN MOLECULAR BIOLOGY, ed. Ausubel et al., Greene Publishing Associates and WileyInterscience, New York, 1988 (with periodic updates); SHORT PROTOCOLS IN MOLECULAR BIOLOGY: A COMPENDIUM OF METHODS FROM CURRENT PROTOCOLS IN MOLECULAR BIOLOGY, 5.sup.th ed., vol. 1-2, ed. Ausubel et al., John Wiley & Sons, Inc., 2002; GENOME ANALYSIS: A LABORATORY MANUAL, vol. 1-2, ed. Green et al., Cold Spring Harbor Laboratory Press, Cold Spring Harbor, N.Y., 1997; CELLULAR AND MOLECULAR IMMUNOLOGY, 4.sup.th ed. Abbas et al., WB Saunders, 1994.

### EXAMPLES

Illustrative Examples are presented below. They are exemplary and non-limiting.

### Example 1: The Use of Activated Charcoals to Remove Impurity During the Purification of Plasmid DNA

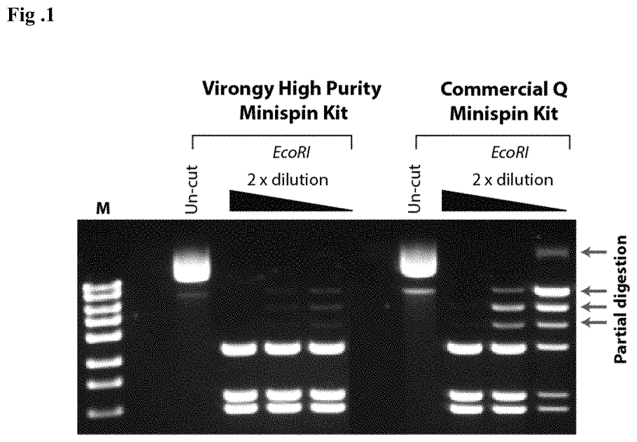

As exemplified in FIG. 1, activated carbon particles were used to remove impurities during the purification of plasmid DNA from E. coli. An equal amount of overnight bacterial cell culture (1.5 ml) was purified using a commercial Q miniSpin plasmid purification kit, or using a similar kit from Virong LLC (Virongy high purity miniSpin plasmid purification kit) in which activated charcoal particles were added to bacterial cell lysates during purification. Following plasmid DNA purification, an equal amount of plasmid DNA, purified with the commercial Q miniSpin kit or with the Virongy miniSpin kit was digested with different amounts of E.coRI that was serially diluted (1:2 dilution). Following digestion, DNA was resolved by agarose gel electrophoresis. As shown, activated charcoal treatment leads to greater plasmid DNA purity that can be readily digested by E.coRI. At low E.coRI concentrations, the presence of impurities in the DNA purified from the Q miniSpin kit inhibits E.coRI activity, resulting in partial digestions.

### Example 2: The Use of Activated Charcoals to Remove Impurity During the Purification of Plasmid DNA

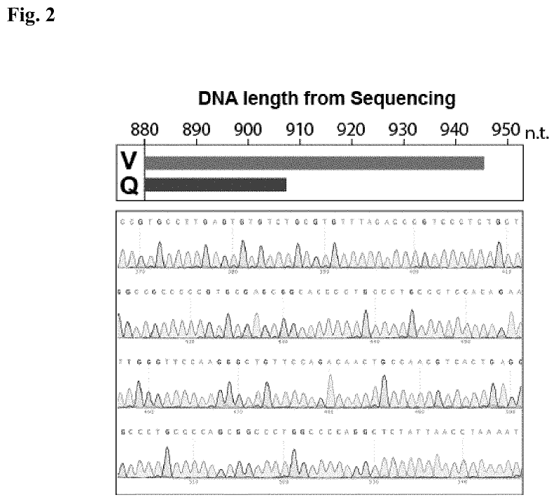

As exemplified in FIG. 2, activated carbon particles were used to remove impurities during plasmid DNA purification, leading to high purity DNA that can be sequenced with longer nucleotide read. An equal amount of overnight bacterial cell culture (1.5 ml) was purified using a commercial Q miniSpin plasmid purification kit, or using a similar kit from Virong LLC (Virongy high purity miniSpin plasmid purification kit) in which activated charcoal particles were added to bacterial cell lysates during plasmid purification. Following plasmid DNA purification, an equal amount of plasmid DNA, purified with the commercial Q miniSpin kit or the Virongy miniSpin kit, was used as the template for DNA sequencing analysis. DNA purified with activated charcoal particles gave a sequence read of 20 to 40 nucleotide longer than DNA purified with the commercial Q miniSpin kit (Average sequenced DNA length from activated charcoal/Virongy MiniSpin kit: 919±24.77; Average sequenced DNA length from Q MiniSpin kit: 898±19.45; p=0.00196)

### Experimental Procedure for Using Activated Charcoals to Facilitate Plasmid DNA Purification

1) In a microcentrifuge tube that holds at least 1.8 ml, add activated charcoal particles 1.5 ml of an overnight bacterial culture, and mix thoroughly by inverting.
2) Centrifuge the bacterial culture and activated charcoal mixture at maximum speed (13,000-16,000 rpm) for 60 seconds. Carefully discard the supernatant without disturbing the pellet. Resuspend the pellet by adding 150 μl of Buffer A (50 mM Tris.HCl, 10 mM EDTA, 100 ug/ml RNase A) into the microcentrifuge tube and pipetting vigorously.
3) Add 150 μl of Buffer B (200 mM NaOH, 1% SDS) to the resuspended pellet mix, and lyse bacterial cells by inverting the tube 8 times.
4) Neutralize the lysis reaction by adding 300 μl of Buffer C (4.2M Gu-HCl, 0.9M Potassium acetate, pH4.8) to the microcentrifuge tube and inverting the tube 8 times.
5) Centrifuge at maximum speed for 10 minutes. A compact black pellet will form.
6) Decant the supernatant, which contains the plasmid DNA, into a spin column assembly. Centrifuge for 30 seconds and discard flow-through. The plasmid DNA will bind to the column.
7) Add 650 μl of Buffer D (10 mM Tris.HCl, pH7.5, 80% ethanol) to the spin column to wash away contaminants. Centrifuge for 30 seconds at maximum speed. Discard the flow-through.
8) Remove any residual buffer by centrifuging the spin column at maximum speed for 30 seconds. Discard the flow-through.
9) Elute plasmid DNA by placing the spin column into a clean 1.5 ml collection tube, and adding 50 μl of Buffer E (TE buffer or H20). Incubate for 60 seconds at room temperature. Then centrifuge at maximum speed for 60 seconds.
10) Store eluted plasmid DNA at 4° C.

1-4. (canceled)

5. A method of formulating activated carbon for use in nucleic acid extraction, comprising
(a) harvesting cells through centrifugation;
(b) resuspending the cells in a lysis buffer;
(c) adding activated carbon particles either before or after cell lysis;
(d) removing cell debris and activated carbon particles by centrifugation;
(e) applying DNA or RNA-containing supernatant to a DNA or RNA-binding column;
(f) washing the column with washing buffer to remove cellular contaminants; and
(g) eluting DNA or RNA with a buffer or with H₂O.

6. The method of claim 5, wherein the buffer is TE buffer.

7. The method of claim 5, wherein the cells are chosen from bacterial cells, bacterial cell cultures, or biological samples containing bacterial cells.

8. The method of claim 7, wherein the method results in the purification of bacterial genomic DNA or bacterial plasmid DNA.

9. The method of claim 5, wherein the cells are chosen from animal cell cultures, animal tissue samples, and animal fluid samples.

10. The method of claim 9, wherein the animal fluid samples are chosen from blood and sperm.

11. The method of claim 10, wherein the method results in the purification of genomic DNA, cytoplasmic DNA or RNA.

12. The method of claim 11, where the cytoplasmic DNA is mitochondrial DNA.

13. The method of claim 9, wherein the method results in the purification of genomic DNA, cytoplasmic DNA or RNA.

14. The method of claim 13, where the cytoplasmic DNA is mitochondrial DNA.

15. A kit comprising activated carbon, and one or more of DNA or RNA extracting buffers, DNA or RNA binding buffers, DNA or RNA-binding columns, column washing buffers, and DNA elution buffers.
